(19) United States
(12) Patent Application Publication
(10) Pub. No.: US 2011/0100865 A1
(43) Pub. Date: May 5, 2011

(54) Hygienic Container

(76) Inventors: Jayne Alyce Brink, Escondido, CA (US); Barry Kevin Brink, Escondido, CA (US)

(21) Appl. No.: 12/912,403

(22) Filed: Oct. 26, 2010

Related U.S. Application Data
(60) Provisional application No. 61/257,455, filed on Nov. 2, 2009.

(51) Int. Cl. A61B 19/02; B65D 69/00

(52) U.S. Cl. 206/581; 206/63.5

(57) ABSTRACT

A hygienic container that stores various personal care items in a clean, hygienic, and inconspicuous manner is disclosed. The hygienic container includes an inner compartment accessible by means of a hinged door. The inner compartment has multiple drain holes from which fluids can drain as well as various means for storing personal items such as toothbrushes, toothpaste, dental floss, and charging bases. An opening in the back of the inner compartment allows access for electrical cords used in charging various personal items. One or more surfaces or parts thereof comprise magnets such that magnetic items and/or items that include ferrous metals can be attached thereto. The hygienic container also includes feet that lift the bottom surface off the ground, thereby enabling drainage from and air circulation through the inner compartment. A UV germicidal light turns on after the door is closed to disinfect items contained within the hygienic container.

### CROSS REFERENCE TO RELATED APPLICATIONS

This is a non-provisional application of U.S. Provisional Pat. App. No. 61/257,455 filed on Nov. 2, 2009, the entirety of which is hereby incorporated by reference.

### STATEMENT REGARDING FEDERALLY SPONSORED RESEARCH OR DEVELOPMENT

This invention was not federally sponsored.

### BACKGROUND OF THE INVENTION

This invention relates to the general field of devices for improving hygiene, and more specifically toward a hygienic container that stores various personal care items in a clean, hygienic, and inconspicuous manner. The hygienic container includes an inner compartment accessible by means of a hinged door. The inner compartment has a plurality of drain holes from which fluids can drain as well as various means for storing personal items such as toothbrushes, toothpaste, dental floss, and charging bases. An opening in the back of the inner compartment allows access for electrical cords used in charging various personal items. One or more surfaces or parts thereof comprise magnets such that magnetic items and/or items that include ferrous metals can be attached thereto. The hygienic container also includes feet that lift the bottom surface off the ground, thereby enabling drainage from and air circulation through the inner compartment.

Personal hygiene is an important part of daily life. It keeps us in good health and promotes a positive appearance. Dental hygiene, in particular, includes daily tasks that keep teeth and gums clean and healthy. These tasks generally require the use of specialized tools and substances, such as toothbrushes, toothpaste, floss, and mouthwash. Since the major goal of these tools and substances is to clean, it is important that they themselves are kept clean.

Leaving a toothbrush out on a counter is not only visually unappealing, but can also make it dirty. Dust, hair, viruses, bacteria, and other contaminants can collect on toothbrushes haphazardly left about a bathroom. Even if these toothbrushes are placed in a container, fluids from a recently used toothbrush can collect and, after a period of time, can lead to bacterial growth. Floss, toothpaste, and mouthwash should be readily available to a user to use throughout the day; however, this can also lead to a cluttered and unkempt bathroom.

Power toothbrushes, those that vibrate and/or otherwise move the bristles, are generally battery powered and often need to be recharged by means of a base station. However, the base station and associated cord add to an often already cluttered bathroom counter. The toothbrush is usually charged in an upright position, with the head of the brush on top. After use, fluids left on the head of the brush drip down onto the remainder of the brush and/or base station causing them to become dirty.

Thus there has existed a long-felt need for a hygienic container that not only organizes personally hygienic items in an appealing manner, but also keeps them clean and dry. It must allow for easy access to the objects within the container such that the performance of daily hygiene tasks is not hindered. There is also a need for hygienic container that can properly house powered toothbrushes in a hygienic fashion.

### SUMMARY OF THE INVENTION

The current invention provides just such a solution by having a hygienic container that stores various personal care items in a clean, hygienic, and inconspicuous manner. The hygienic container includes an inner compartment accessible by means of a hinged door. The inner compartment has a plurality of drain holes from which fluids can drain as well as various means for storing personal items such as toothbrushes, toothpaste, dental floss, and charging bases. An opening in the back of the inner compartment allows access for electrical cords used in charging various personal items. One or more surfaces or parts thereof comprise magnets such that magnetic items and/or items that include ferrous metals can be attached thereto. The hygienic container also includes feet that lift the bottom surface off the ground, thereby enabling drainage from and air circulation through the inner compartment. A UV germicidal light turns on after the door is closed to disinfect items contained within the hygienic container.

It is a principal object of the invention to provide a hygienic container that stores personal hygiene tools and substances.

It is a further object of the invention to provide a hygienic container that promotes an environment for personal items that is safe from viruses and bacteria.

It is another object of the invention to provide a hygienic container that aids a user in keeping a bathroom uncluttered.

It is an additional object of the invention to provide a hygienic container that maintains tools and substances in a state that is cleaner than had the tools and substances simply been left out on top of a counter.

It is a final object of this invention to provide a hygienic container that is aesthetically pleasing.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a front view of a hygienic container according to the current invention showing multiple personal care items contained therein.

FIG. 2 is a front view of the hygienic container.

FIG. 3 is a front view of the hygienic container with the container removed.

FIG. 4 is a bottom view of alternative embodiments of the top of the hygienic container.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

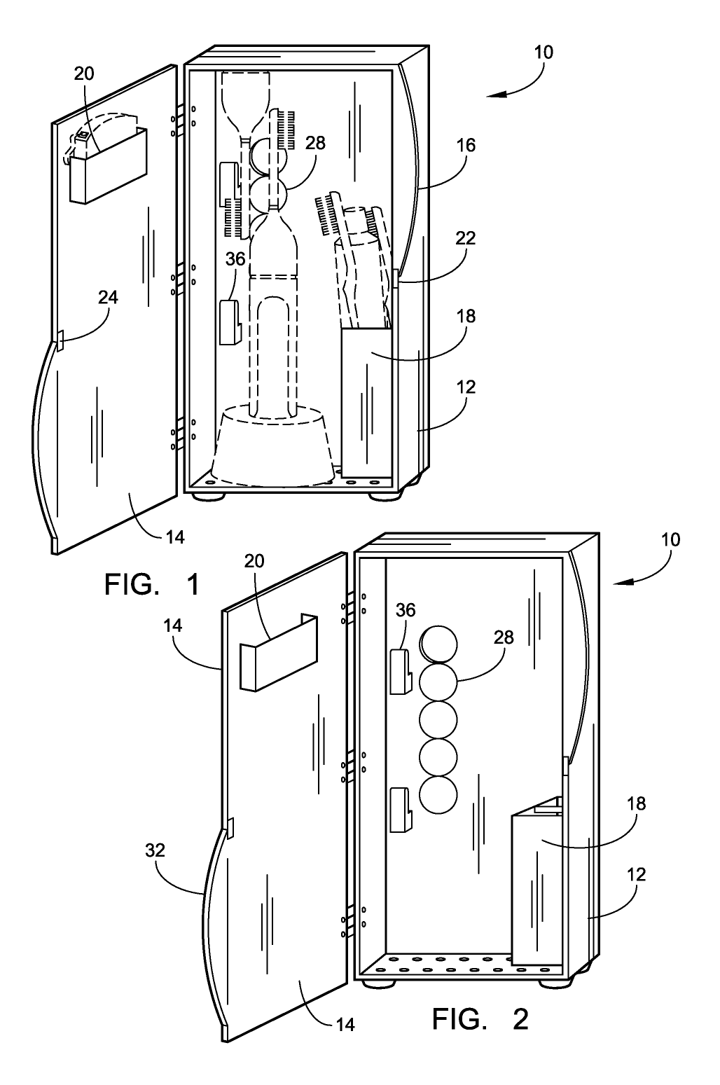

FIG. 1 is a front view of a hygienic container according to the current invention showing multiple personal care items contained therein. The hygienic container 10 includes a door 14 that is attached to the main body by means of hinges. The door 14 encloses an inner compartment within the hygienic container 10 when in a closed position. The door 14 is secured in a closed position by mating a magnet 24 on the door 14 with a magnet 22 on the sidewall 12 of the hygienic container 10. The door 14 includes a dental floss holder 20 to hold dental floss or other similarly sized items. A container 18, or toothbrush holder, is secured to the main body, preferably on one of the sidewalls, whereby a user can place a toothbrush, tube of toothpaste, or other similarly sized object. Curved recess 16 enables users to open door 14 by grasping the upper edge of the door.

FIG. 2 is a front view of the hygienic container. Handle 32 is used to open the door 14 to gain access to the inner compartment of the hygienic container 10. A plurality of openings 28 are located in the back of the hygienic container 10 to allow power cables to be inserted therethrough. Power cables can be used to charge various electronic devices, including electric toothbrushes. The openings 28 also allow for air to circulate within the inner compartment of the hygienic container 10, even when the door 14 is closed. Power cables are often longer than necessary; this extra length of power cable can be wound around hooks 36 for safe and convenient storage.

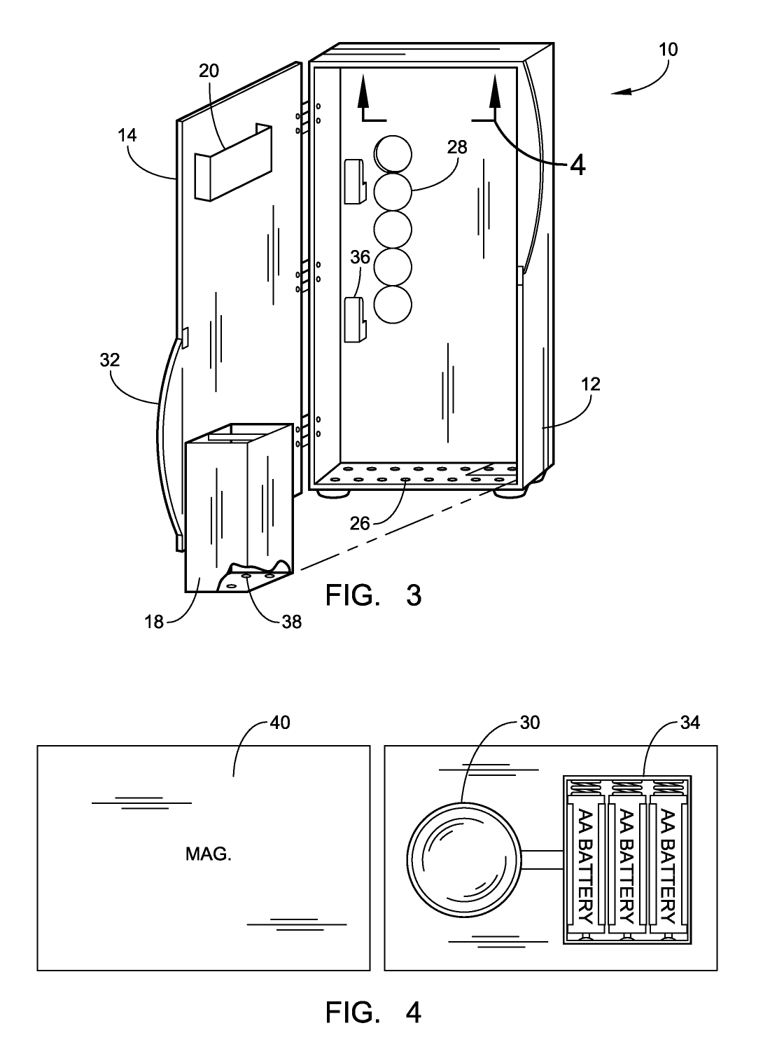

FIG. 3 is a front view of the hygienic container with the container removed. The floor of the inner compartment of hygienic container 10 includes a plurality of drain holes 26. The drain holes 26 allow for excess fluids to drain from the inner compartment as well as to allow for the circulation of air within the inner compartment. The container 18 itself also has a plurality of drain holes 38 to allow for excess fluids to drain from the container 18 into the inner compartment and then from the inner compartment through holes 26 out of the hygienic container 10.

FIG. 4 is a bottom view of alternative embodiments of the top of the hygienic container. The bottom of the top, or roof, of the hygienic container 10 includes a magnet 40. This magnet can be used to affix magnetic and/or ferrous metals thereto. For example, some toothbrushes include magnetic bases, and thus can be hung upside down for storage. Such a configuration is shown in FIG. 1. To this end, brushes with magnetic ends can hang upside down whereby excess fluids on the head of the brush falls down to the floor and through the drain holes 26. This feature greatly reduces the amount of fluids left on the brush and therefore reduces the chances of bacterial growth.

The bottom of the roof of the hygienic container also has a light and a battery case. The light 30 is electrically connected to and powered by one or more batteries in a battery compartment 34 that is preferably adjacent to the light 30. An ultraviolet (UV) germicidal light is used to help disinfect any personal items contained within the hygienic container. The light can be turned on and off by means of a switch. This switch is preferably integrated into the door and is activated when the door closes.

The UV germicidal light preferably illuminates only when the door is closed, and then only for a set period of time. To this end, when the door closes and the switch is activated, the germicidal light illuminates. Then, after a brief period of time, such as 60 seconds, the UV germicidal light turns off. In this manner, the personal items are sanitized after being placed within the hygienic container without unnecessarily irradiating the user or expending unwarranted power from the batteries. Alternatively, a toggle switch may be incorporated into the hygienic container, such as on the battery compartment 34 itself, whereby a user may turn the UV germicidal light on and off by toggling the switch.

In an alternative embodiment, the light is a non-UV light used to illuminate the inner compartment of the hygienic container. When the door opens, the light turns on, and when the door closes, the light turns off.

The hygienic container according to the current invention also includes four feet, one located at each corner of the base of the hygienic container. The feet, preferably made from rubber, provide stability to the main body as well as reduce the chance of slippage that may occur on smooth surfaces. Further, the feet raise the base of the hygienic container off of the surface below thereby allowing air to pass underneath and fluids to drain from the drain holes in the floor. A fan can be included with the hygienic container to improve air circulation within and through the inner compartment.

A portable version is shown and described in FIG. 1; however, it is contemplated that a permanently affixed embodiment is possible without departing from the scope of the current invention. It could be permanently secured to the surface of a counter or set within a wall of a home or a recreational vehicle. Instead of openings for cords, an outlet could be set in such an area.

The surface of the hygienic container can be solid, or made from an underlying material and an external façade. The materials from which the hygienic container can be made from include granite, marble, metal, copper, bamboo, rattan, plastic, wood, vinyl, canvas, mirrored surfaces, and/or injected foam rubber. The hygienic container, through an applied façade, can also be made to look like the previously listed materials even though a substantial portion of the hygienic container is made from a different, preferably less expensive, material.

While the preceding disclosure has focused on items for dental hygiene, it is contemplated that various items can be stored within the hygienic container, including soap, shampoo, makeup, and contact lens cases and solution. Additional components can also be integrated into the main body to improve the hygienic qualities of the container, including UV lights, antimicrobial surfaces, and a water ionizer. A permanent or semi-permanent toothpaste and/or mouthwash dispenser can be affixed within the hygienic container. An LCD screen can be integrated into the hygienic container, preferably into the door, whereby messages and other information can be graphically displayed to the user. Through this or other means, reminders, lists, pictures, and other such data are be displayed to the user. Additionally, the hygienic container can include a speaker through which music or other audio content can be played.

Electronic items of the hygienic container, including the UV lights, other lights, and LCD screen are preferably battery powered, though an electrical plug can also be used to power these items. The batteries are preferably replaceable and rechargeable. If the batteries are rechargeable, then a means of recharging the batteries can be included with the hygienic container. One such means of recharging is by using solar panels. While solar panels run most efficiently in direct sunlight, they are nonetheless capable of producing energy in environments with less incident light, including bathrooms.

In an alternative embodiment, a shower curtain style enclosure can be used instead of the door. In that case, the dental floss holder can be located on a sidewall. In another alternative, two doors can be used, whereby the two doors open outward.

To use the hygienic container of the current invention, a user will open the door using the handle, and will place one or more hygienic items therein. The door is then closed. When desired, a user will open the door and remove one or more hygienic items therein, such as a toothbrush. The user then uses the one or more hygienic items and then returns them to the hygienic container, and closes the door. The hygienic items are then allowed to dry within the hygienic container as well as be hidden and protected therein.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. A hygienic container comprising
a main body, whereby the main body bounds an inner compartment, where the main body comprises a floor, a back wall, two side walls, and a top, where the floor comprises a plurality of holes, where one of the sidewalls comprises a magnet, where the back wall comprises two hooks and a plurality of openings,
a means for accessing the inner compartment, where the means for accessing the inner compartment is a door, where the door comprises a handle, a dental floss holder, and a magnet, where the magnet of the door mates with the magnet of the sidewall to removably secure the door to the sidewall,
a plurality of feet that support the main body, and
a container, where the container removably resides within the inner compartment, where the container comprises a plurality of holes.

2. The hygienic container of claim 1, further comprising a light, where the light is affixed to the top.

3. The hygienic container of claim 2, wherein the light is a ultraviolet germicidal light.

4. The hygienic container of claim 1, wherein the top comprises a magnet.

5. A hygienic container comprising
a main body, whereby the main body bounds an inner compartment, where the main body comprises a floor, a back wall, two side walls, and a top, where the floor comprises a plurality of holes, where the back wall comprises an opening,
a means for accessing the inner compartment, and
a container, where the container removably resides within the inner compartment, where the container comprises a plurality of holes.

6. The hygienic container of claim 5, wherein the means for accessing the inner compartment is a door.

7. The hygienic container of claim 6, wherein the door comprises a handle.

8. The hygienic container of claim 6, further comprising a dental floss holder.

9. The hygienic container of claim 8, wherein the dental floss holder is secured to the door.

10. The hygienic container of claim 6, wherein one of the sidewalls comprises a magnet and the door comprises a magnet, where the magnet of the door mates with the magnet of the sidewall to removably secure the door to the sidewall.

11. The hygienic container of claim 5, wherein the back wall comprises a plurality of openings.

12. The hygienic container of claim 5, wherein the back wall comprises two hooks.

13. The hygienic container of claim 5, further comprising a light, where the light is affixed to the top.

14. The hygienic container of claim 13, wherein the light is an ultraviolet germicidal light.

15. The hygienic container of claim 13, further comprising a battery compartment, where the battery compartment is electrically connected to the light.

16. The hygienic container of claim 5, further comprising a battery compartment.

17. The hygienic container of claim 5, further comprising a plurality of feet that support the main body.

18. The hygienic container of claim 5, wherein the top comprises a magnet.

19. A method of storing hygienic items comprising the steps of:
obtaining a hygienic container, where the hygienic container comprises a main body, whereby the main body bounds an inner compartment, where the main body comprises a floor, a back wall, two side walls, and a top, where the floor comprises a plurality of holes, where one of the sidewalls comprises a magnet, where the back wall comprises two hooks and a plurality of openings, where the hygienic container further comprises a means for accessing the inner compartment, where the means for accessing the inner compartment is a door, where the door comprises a handle, a dental floss holder, and a magnet, where the magnet of the door mates with the magnet of the sidewall to removably secure the door to the sidewall, where the hygienic container further comprises a plurality of feet that support the main body, and where the hygienic container further comprises a container, where the container removably resides within the inner compartment, where the container comprises a plurality of holes;
opening the door of the hygienic container;
placing one or more hygienic items therein; and
closing the door of the hygienic container.

20. The method of claim 18, further comprising the steps of:
once again opening the door of the hygienic container;
removing a hygienic item; and
using the hygienic item.

21. The method of claim 19, further comprising the steps of:
placing the hygienic item back within the hygienic container; and
closing the door;
thereby allowing the hygienic item to dry within the hygienic container.

22. The method of claim 18, further comprising a light, where the light is affixed to the top, where the light is an ultraviolet germicidal light, where the light irradiates the inner compartment for a period of time after the door is closed.
